(19) United States
(12) Patent Application Publication
(10) Pub. No.: US 2017/0248526 A1
(43) Pub. Date: Aug. 31, 2017

(54) QUALITY CONTROL SYSTEM FOR AGRICULTURE PRODUCTS

(71) Applicants: Rolands VINOGRADOVS, Ceraukste pag. (LV); BALTIC AGRO TEHNOLOGIJAS, SIA, Bauska (LV)

(72) Inventor: Rolands VINOGRADOVS, Ceraukste pag. (LV)

(21) Appl. No.: 15/508,934

(22) PCT Filed: Sep. 26, 2014
(86) PCT No.: PCT/IB2014/064850

§ 371 Date: Mar. 6, 2017

(51) Int. Cl. G01N 21/94; G01N 21/3563; B07B 1/36; G01N 21/88; G01N 1/20; G01N 33/02; G01N 21/85; G01N 29/04

(52) U.S. Cl. CPC G01N 21/94; G01N 21/85; G01N 21/3563; G01N 29/04; G01N 21/8851; G01N 1/20; G01N 33/025; B07B 1/36; G01N 2021/8854; G01N 2021/8592; B07B 2201/04

(57) ABSTRACT

Invention refers to quality control system for detection and identification of insects, pests and the like in such product like cereals, seed, grains and the like. The Quality control system comprises a sample collecting device (1) for collecting a sample in pre-determined amount from a product (P) to be tested, a self-cleaning cyclone filter (2) for cleaning the sample, a vibro separator (3) for separation of the sample in pre-determined sizes or grades, a heating chamber (4) for heating the sample separated in the vibro separator. The system further comprises an identification device (5) for detection and identification of live insects, pests and the like in said sample.

Invention refers to quality control system, especially to quality control systems detecting a presence of insects, pests and the like in such product like cereals, seed, grains and the like.

Early detection and disinfection of supplies eliminates product damage by pests. A product to be controlled is selected from the group of the following products: corn, rice, wheat, barley, sorghum, millet, oats, rye, triticale, buckwheat, white mustard, black mustard, brown mustard, hemp, cumin, poppy, rape, sunflowers, flax, safflower, beans, peas, Turkish peas, lentils, soybeans, peanuts, legume seed flour products.

Quality control of cereals, seed, grains and the like is performed by manually collecting a sample and by visual analysis of collected sample. Control of said products usually is performed during bulk cargo loading, unloading and handling. Said quality control includes detection and identification of live pests, larvae, insects and the like.

In this way, previous quality control systems do not adequately provide an efficient, reliable and fast quality control of tested sample. Such a process always will be influenced by human factor. Therefore, it is necessary to develop a quality control system which is efficient, fast and reliable in detection and identification of live pests, insects in cereals, seed, grains and the like.

The aim of the invention is reached by designing a quality control system for controlling the cereals, seeds, grains and the like. The quality control system comprising a sample collecting device for collecting a sample in pre-determined amount from a product to be tested, a self-cleaning cyclone filter for cleaning the sample, a vibro separator for separation of the sample in pre-determined sizes, a heating chamber for heating the sample separated in the vibro separator. The system further comprises a noise sensor, movement scanner for identifying a presence of life insects, pests and the like in said sample, an infrared spectrometer for detection and identification of life insects, pests and the like in said sample, a thermo visor for detection and identification of life insects, pests and the like in said sample, and an image recognition device for detection and identification of life insects, pests and the like in said sample.

The noise and movement scanner, the infrared spectrometer, the thermo visor and the image recognition device can be incorporated in an identification apparatus.

At the final stage of the system it further comprises a disinfection tank for reception and disinfection of processed samples. If live insects, pests and the like are detected, the sample is moved to the disinfection tank. The same tank can be used to disinfect all cargo, from which the sample were taken.

The identification apparatus further comprises an ultrasound sensor for detection of life insects, pests and the like in said sample. Said ultrasound sensor can be installed in any place in the system according to the needs of system user. Installation of said sensor gives additional security that the insects, pests and the like will be detected.

Another embodiment of the invention is a quality control system comprising a sample collecting device for collecting a sample in pre-determined amount from a product to be tested, a self-cleaning cyclone filter for cleaning the sample, a vibro separator having three stages for separation of the sample in three samples. A first sample separated in the first stage flows back through a discharge opening to the product flow. In the first stage, impurities are separated. In the second stage a second sample is separated, which can comprise live insects, pests and the like. In the third stage a third sample is separated, wherein the third sample has crops or any other agricultural product with smaller grade than in the second sample. The second and the third sample is transported to the heating chamber for heating said samples separately. After the heating, the third sample as well as the second sample are transported to the noise and movement scanner for detection a presence of live insects, pests and the like. For each sample there is separate noise and movement scanner.

The second sample is further transported to the infrared spectrometer, the thermo visor and the image recognition device. If the presence of live insects, pests and the like is detected the second sample is sent to the disinfection tank. If the presence of live insects, pests and the like is not detected the second sample is sent to the outflow from the system or back to the flow of basic product.

If the presence of live insects, pests and the like is detected in the third sample in the noise and movement scanner then the third sample is sent for further investigation to the thermo visor and to the image recognition device and subsequently to the disinfection tank. If the presence was not detected in the noise and the movement scanner, then the third sample is discharged through the discharge opening back to the product conveyor or tank through pneumatic line or transporter.

The collecting device is configured to collect a sample of product, which is transported through pipeline or which is accommodated in a tank. The collecting device can comprise pneumatic system configured to collect a predetermined amount of the product to be tested. The collecting device can be a frame configured to collect the samples while the product flows through the frame. After collecting of the sample it is transported to the self-cleaning cyclone filter that is configured to clean the sample from dusts and other impurities.

After the cleaning the sample is conveyed to the vibro separator, that is configured to separate the samples of different nature, grades and sizes, wherein said product passes through a series of screen, preferable at least three screens. The vibro separator to be used can be three-level, three-dimensional motion vibro separator with interchangeable separators with self-cleaning and unloading functions. The vibro separator can be in at least two stages, preferably in three stages. The system can comprise more than one vibro separator to increase an overall efficiency. For example, the system can comprise two separators connected parallel, doubling the efficiency.

Said sensor units are interchangeable and can be arranged in different manner from previous mentioned. For example, the first identification unit can be the infrared spectrometer instead of the noise and movement scanner. Position of said sensor units depend on configuration of the system according to the needs of a user.

A rotor conveyor can be installed in the identification apparatus for transportation of sample from one control unit to another one. Accordingly, a sample is loaded in a rotor conveyor and then is transported from the noise and movement scanner to the infrared spectrometer and later on to the thermo visor and the image recognition device for detection and identification of life insects, pests and the like in said sample. The movement of samples can be accomplished by belt conveyor and by any other transportation means configured to convey a sample to be tested from one to another unit of the system.

The present invention further comprises a method for detection and identification of live insects, pests and the like using said quality control system. The method includes the following steps: collecting a sample of a product using a collecting device, cleaning the sample in a self-cleaning cyclone filter, separation of the sample in pre-determined sizes or grades using a vibro separator, heating of each separated sample in a heating chamber, detecting a presence of live insects, pests and the like in said sample by a noise and movement scanner, detecting and identifying of live insects, pests and the like in said sample using infrared spectrometer, a thermo visor and an image recognition device.

After detection and identification of live insects, pests and the like the second and the third samples are sent to disinfection tank or discharged through a discharge opening.

Detection and identification of a live insects, pests and the like in said sample is performed by using a noise sensor, a movement scanner, a noise or movement scanner, an infrared spectrometer, a thermo visor, an image recognition device or an ultrasound sensor.

After detection and identification of live insects, pests and the like each sample is sent to disinfection tank or discharged through a discharge opening. Usually, when live insects, pests and the like are detected the sample is sent to the disinfection tank. In result all cargo can be sent to the disinfection tank. The disinfection tank is configured to keep a constant disinfection environment. When there is no detection, the sample is sent to the discharge opening to return it into a product tank.

At least one sample, where the presence of insects were detected, can be discharged through a discharge opening to a compartment configured to store a collected sample. This feature is very useful to preserve the tested sample for further quality control or for documentation of test results.

The aspects of the present invention will become apparent and more readily appreciated from the following description of embodiments, taken in conjunction with the accompanying drawings in which:

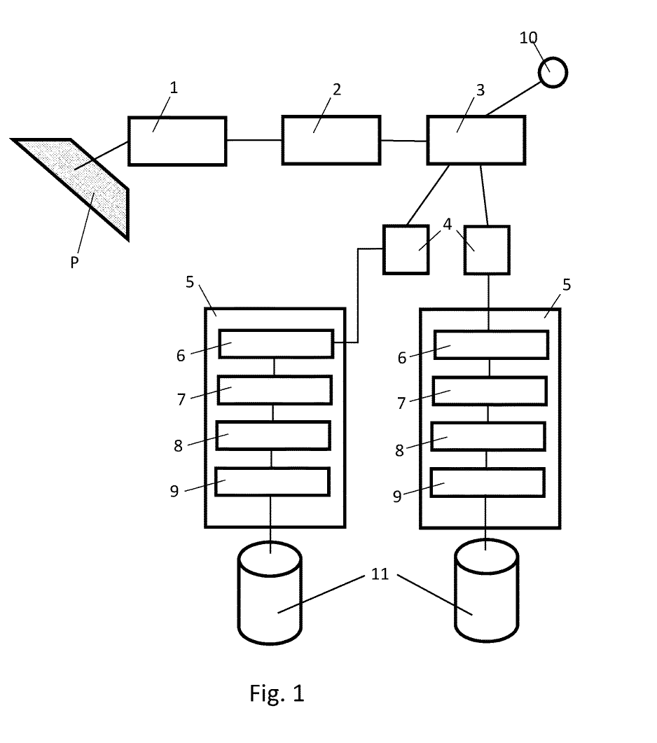

FIG. 1 is a block diagram illustrating a quality control system;

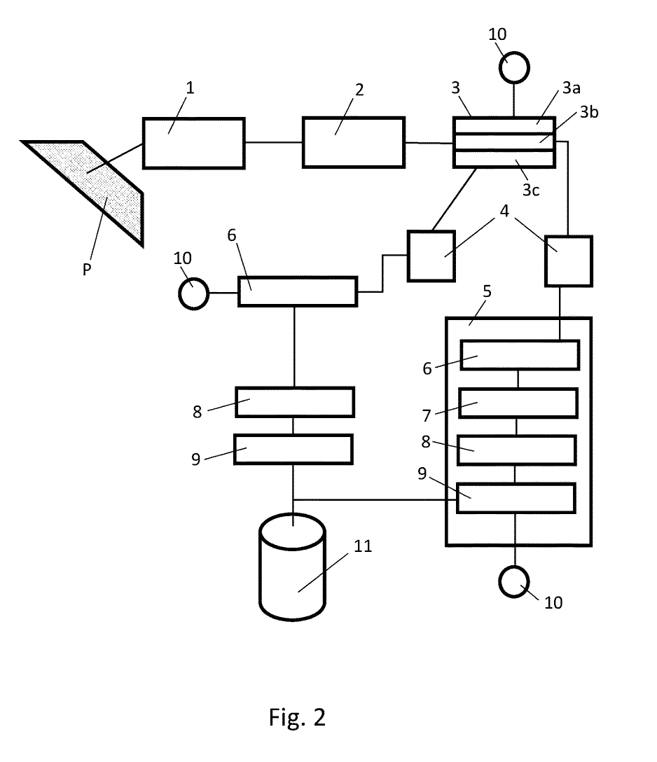

FIG. 2 is a block diagram illustrating an embodiment of a quality control system;

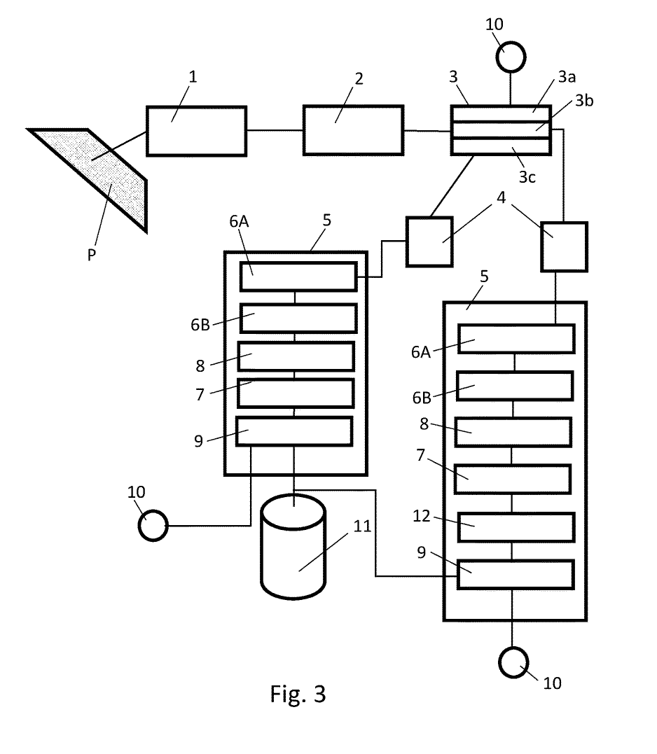

FIG. 3 is block diagram illustrating another embodiment of a quality control system.

A quality control system (FIG. 1) for controlling of cereals, seeds, grains and the like, comprises a sample collecting device 1 for collecting a sample in pre-determined amount from a product P to be tested, a self-cleaning cyclone filter 2 for cleaning the sample and a vibro separator 3 for separation of the sample in pre-determined sizes or grades. The following example in FIG. 1 illustrates the three stage vibro separator 3. The samples separated at a first stage are discharged to a discharge opening 10. The samples in the first stage are impurities that cannot be used for testing. The samples separated in the second stage and in the third stage are the samples to be tested. Said samples differ in the grain size.

After separation the second and the third sample are transported to a heating chamber 4 for heating the sample separated in the vibro separator. Each heated sample is then sent to an identification apparatus 5 for detection and identification of live insects, pests and the like in said sample.

Said identification apparatus 5 comprises a noise and movement scanner 6 for detection a presence of live insects, pests and the like in said sample, an infrared spectrometer 7, a thermo visor 8 for detection and identification of live insects, pests and the like in said sample, and an image recognition device 9 for identification of live insects, pests and the like in said sample. Accordingly, a user of the system will be able to recognize the detected insect, pest or the like. Afterwards said tested samples are transported to a disinfection tank 11 for disinfection.

Another embodiment of a quality control system (FIG. 2) comprises a sample collecting device 1 for collecting a sample in pre-determined amount from a product P to be tested, a self-cleaning cyclone filter 2 for cleaning the sample and a vibro separator 3 for separation of the sample in pre-determined sizes or grades. The vibro separator 3 comprises three stages 3a, 3b and 3c. The samples separated at a first stage 3a are discharged to a discharge opening 10. The samples in the first stage are different impurities that cannot be used for testing. The samples separated in the second stage 3b and in the third stage 3c are the samples to be tested. Said samples differ in the grain sizes. After separation, the second and the third sample are transported to a heating chamber 4 for heating the sample.

The third samples are sent to the noise and movement scanner 6 for detection a presence of live insects, pests and the like in said sample. If the presence is detected the sample is sent to the thermo visor 8 and the image recognition device 9 for further investigation and afterwards to the disinfection tank 11 for disinfection. If in the noise and movement scanner 6 the presence of live insects, pest or the like is not detected, then the sample is sent to the discharge opening 10.

The second samples are sent to an identification apparatus 5 for detection and identification of live insects, pests and the like in said sample.

Said identification apparatus 5 comprises a noise and movement scanner 6 for detection a presence of live insects, pests and the like in said sample, an infrared spectrometer 7, a thermo visor 8 for detection and identification of live insects, pests and the like in said sample, and an image recognition device 9 for identification of live insects, pests and the like in said sample. If the presence of live insects, pest or the like is detected and identified the sample is sent to the disinfection tank 11 for disinfection. If the presence is not detected, then the sample is sent to the discharge opening 10. At least one sample, where the presence of insects were detected, can be discharged through a discharge opening 10 to a compartment (not shown in figure) configured to store a collected sample. At least one sample, where the presence of insects were detected, can be discharged through a discharge opening 10 to a compartment (not shown in figure) configured to store a collected sample.

Another embodiment of a quality control system (FIG. 3) comprises a sample collecting device 1 for collecting a sample in pre-determined amount from a product P to be tested, a self-cleaning cyclone filter 2 for cleaning the sample and a vibro separator 3 for separation of the sample in pre-determined sizes or grades. The vibro separator 3 comprises three stages 3a, 3b and 3c. The samples separated at a first stage 3a are discharged to a discharge opening 10. The samples in the first stage are impurities that cannot be used for testing. The samples separated in the second stage 3b and in the third stage 3c are the samples to be tested. Said samples differ in the grain sizes. After separation the second and the third sample are transported to a heating chamber 4 for heating the sample.

Each third sample is sent to identification apparatus 5. The identification apparatus 5 comprises a noise sensor 6A and a movement scanner 6B, the thermo visor 8, the infrared spectrometer 7 and the image recognition device 9. If the presence of live insects, pest or the like is detected and identified the sample is sent to the disinfection tank 11 for disinfection. If the presence is not detected, then the sample is sent to the discharge opening 10.

Each second sample is sent to identification apparatus 5. Said identification apparatus 5 comprises a noise sensor 6A and a movement scanner 6B, the thermo visor 8, the infrared spectrometer 7, an ultrasound sensor 12 and the image recognition device 9. If the presence of live insects, pest or the like is detected and identified the sample is sent to the disinfection tank 11 for disinfection. If the presence is not detected, then the sample is sent to the discharge opening 10. At least one sample, where the presence of insects were detected, can be discharged through a discharge opening 10 to a compartment (not shown in figure) configured to store a collected sample.

The present invention is not limited to the above-described embodiments. It is apparent to one who has an ordinary skill in the art that there may be many modifications and variations within the scope of the invention as defined by the appended claims.

Explanation of main parts of the drawings:
1—a sample collecting device;
2—a self-cleaning cyclone filter;
3—a vibro separator;
3a—a first stage of the vibro separator;
3b—a second stage of the vibro separator;
3c—a third stage of the vibro separator;
4—a heating chamber;
5—an identification apparatus;
6—a noise and movement scanner;
6A—a noise sensor;
6B—a movement scanner;
7—infrared spectrometer;
8—thermo visor;
9—an image recognition device;
10—discharge opening;
11—disinfection tank;
12—ultrasound sensor;
P—product to be controlled.

1. A Quality control system for controlling of cereals, seeds, grains and the like, comprising
a sample collecting device (1) for collecting a sample in pre-determined amount from a product (P) to be tested,
a self-cleaning cyclone filter (2) for cleaning the sample,
a vibro separator (3) for separation of the sample in pre-determined sizes or grades,
a heating chamber (4) for heating the sample separated in the vibro separator,
wherein said system further comprises a movement scanner (6B) for detection a presence of live insects, pests and the like in said sample,
an infrared spectrometer (7) for detection and identification of live insects, pests and the like in said sample,
a thermo visor (8) for detection and identification of live insects, pests and the like in said sample,
an image recognition device (9) for identification of live insects, pests and the like in said sample.

2. The quality control system according to claim 1, wherein the system further comprises a noise sensor (6A) for detection a presence of live insects, pests and the like in said sample.

3. The quality control system according to claim 1, wherein the noise sensor (6A) and the movement scanner (6B) are combined as a noise and movement scanner (6).

4. The quality control system according to claim 1 wherein the identification apparatus (5) further comprises an ultrasound sensor (12) for detection of live insects, pests and the like in said sample.

5. The quality control system according to claim 1 , wherein the noise sensor (6A), the movement scanner (6B), the infrared spectrometer (7), the thermo visor (8), the image recognition device (9) and ultrasound sensor (12) are incorporated in an identification apparatus (5).

6. The quality control system according to claim 1, wherein it further comprises disinfection tank (11) connected to the final stage of the system for disinfection of processed samples.

7. The quality control system according to claim 1, wherein the vibro separator (3) comprises at least three stages (3a, 3b, 3c) for separation of a sample in three samples,
wherein a first sample separated in the first stage (3a) flows to the outflow (10) of the system, a second sample separated in the second stage (3b) and a third sample separated in the third stage (3c) flow to a heating chamber (4) configured to heat said samples.

8. Method for detection and identification of live insects, pests and the like using said quality control system, wherein the method includes the following steps:
collecting a sample of a product (P) using a collecting device (1),
cleaning the sample in a self-cleaning cyclone filter (2),
separation of the sample in pre-determined sizes or grades using a vibro separator (3),
heating of each separated sample in a heating chamber (4),
detecting and identifying of live insects, pests and the like in said sample.

9. Method according to claim 8, wherein the step for detecting and identifying of a live insects, pests and the like in said sample is performed by using a noise sensor (6A), a movement scanner (6B), a noise or movement scanner (6), an infrared spectrometer (7), a thermo visor (8), an image recognition device (9) or an ultrasound sensor (12).

10. Method according to the claim 8, wherein after detection and identification of live insects, pests and the like each sample is sent to disinfection tank (11) or discharged through a discharge opening (10).
